(19) United States
(12) Patent Application Publication
(10) Pub. No.: US 2010/0048492 A1
(43) Pub. Date: Feb. 25, 2010

(54) COMPOSITION FOR THE PREVENTION AND/OR TREATMENT OF DISEASES ASSOCIATED WITH TNF AND/OR IL-12 OVEREXPRESSION

(76) Inventors: Valérie Quesniaux Ryffel, Saint-Denis En Val (FR); Germain Puzo, Auzeville (FR); Jérôme Nigou, Auzeville Tolosane (FR); Martine Gilleron, Auzeville Tolosane (FR)

Correspondence Address: HARNESS, DICKEY & PIERCE, P.L.C., P.O. BOX 828, BLOOMFIELD HILLS, MI, 48303, US

(21) Appl. No.: 12/515,450

(22) PCT Filed: Nov. 20, 2007
(86) PCT No.: PCT/FR2007/001898

§ 371 Date: Sep. 11, 2009

(30) Foreign Application Priority Data
Nov. 20, 2006 (FR) 0610136

(51) Int. Cl. A61K 31/7028; C07H 11/04; A61P 29/00

(52) U.S. Cl. 514/25; 536/17.1

(57) ABSTRACT

The present invention relates to a pharmaceutical composition comprising at least one compound of formula (I):

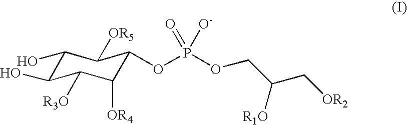

or one of its pharmaceutically acceptable salts in which R₁, R₂ and R₃ are independently a hydrogen or an R₇—CO— group where R₇ is an alkyl, alkene or alkyne group, linear, branched or cyclic, comprising 2 to 24 carbon atoms; R₄ is a hydrogen atom or a mannosyl group substituted in position 6 by an R₆ residue chosen from the group comprising a hydrogen atom and an R₇—CO— group; and R₅ is chosen from the group comprising a hydrogen atom, a mono-, di-, tri-, tetra- and penta-mannosyl; and the use of such a composition for manufacturing a medication intended for the prevention or treatment of an illness associated with the over-expression of TNF and/or IL-12 in a subject.

### CROSS-REFERENCE TO RELATED APPLICATIONS

This application is a National Phase Entry of International Application No. PCT/FR2007/001898, filed Nov. 20, 2007, which claims priority to French Patent Application No. 06/10136, filed Nov. 20, 2006, both of which are incorporated herein by reference.

### BACKGROUND AND SUMMARY

The present invention concerns the field of prevention or treatment of illnesses associated with the over-expression of TNF and/or IL-12 in a subject.

The incidence of inflammatory illnesses, such as rheumatoid arthritis or Crohn's disease is continuously increasing in developed countries, in particular in Europe. For these pathologies, TNF and IL-12 constitute key effectors. Interleucin-12 is a cytokine having a unique structure and pleiotropic effects (Kobayashi et al., J. Exp. Med., vol. 170, p: 827-845., 1989 ; SEDER et al., Proc. Natl. Acad. Sci. USA, vol. 90, p: 10188-10192, 1993; LING et al., J. Immunol., vol. 154, p: 116-127, 1995; Podlaski et al., Arch. Biochem. Biophys., vol. 294, p: 230-237, 1995). This consists of two sub-units (p40 and p35) forming activating heterodimers or inhibiting p40 homodimers. IL-12 is mainly produced by macrophages and monocytes essentially following an activation of diverse origins, endogenous or exogenous, in particular by microorganisms, intracellular parasites, bacteria or bacterial products. Functional studies have shown that IL-12 stimulates the cytolytic activity of NK (Natural Killer) cells and macrophages. Finally, IL-12 fulfils a central role in the differentiation of T cells of the Th1 type and allows induction of the production of IFN-γ.

TFNα is a cytokine secreted by monocytes and macrophages in response to endotoxins or other stimuli. TNFα corresponds to a soluble homotrimer, the protein sub-units of which have 17 kDa (SMITH et al., J. Biol. Chem., vol. 294, p: 6951-6954, 1987). For reviews on TNF, see BEUTLER et al., (Nature, vol. 320, p: 584, 1986), OLD (Science, vol. 230, p: 630, 1986), and LE et al. (Lab. Invest., vol. 56, p: 234). However, cells other than monocytes and macrophages are liable to produce TNFα. By way of example, non-monocyte human cell lines produce TNF (RUBIN et al., J. Exp. Med., vol. 164, p: 1350, 1986; SPRIGGS et al., Proc. Natl. Acad. Sci. USA, vol. 84, p: 6563, 1987). TNF causes a pro-inflammatory reaction that results in tissue damage, such as the induction of a pro-coagulant activity in the endothelial vascular cells (POBER et al., J. Immunol., vol. 136, p: 1680, 1986), an increase in the adhesion of neutrophiles and lymphocytes (POBER et al., J. Immunol., vol. 138, p: 3319, 1987), and stimulation of the release of platelet activating factor by macrophages, neutrophiles and endothelial vascular cells (CAMUSSI et al., J. Exp. Med., vol. 166, p: 1390, 1987).

In order to treat various inflammatory illnesses, namely rheumatoid arthritis, Crohn's disease and psoriasis, various “anti-TNF” therapies have been developed (SCHREIBER et al., 2001) and the number of patients treated in the world with these therapies already reaches one million, mainly in Europe and the USA. The majority of these therapies use antibodies directed against TNF such as those described in the patent U.S. Pat. No. 5,698,195. By way of antibodies directed against TNF and used in therapy, HUMIRA® (ABOTT), CDP-870 (UCB Pharma), AFELIMOMAB® (KNOLL Gmbh), Infliximab® (Centocor) and Remicade® (Shering-Plough) can be cited.

However, these various treatments have revealed undesirable effects such as an increase in the tendency to develop tuberculosis and opportunistic infections (MOHAN et al., Curr. Opin. Rheumatol., vol. 15, p: 179-184, 2003). Thousands of cases have been reported in patients treated by anti-TNF therapies (ASKLING et al., Arthritis Rheum, vol. 52, p: 1986-1992, 2005), the majority being atypical tuberculoses, difficult to diagnose and corresponding to cases of disseminated and extrapulmonary tuberculosis, and probably related to the reactivation of a latent chronic infection (MOHAN et al., Clin. Infect. Dis., vol. 39, p: 295-299, 2004). Thus up to 1-2% of patients treated by anti-TNF are liable to develop tuberculosis and, because of the reduction in the cost of anti-TNF therapies, the number of patients affected may increase. Though the majority of these infections correspond to the reactivation of a latent infection, close on 30% of the cases correspond to primo infections. Finally, these anti-TNF treatments for patients having an antecedent of tuberculosis infection require antibiotic treatment for close on 9 months (KEANE, Rheumatology, Oxford. 2005).

Treatments directed against IL-12 are at an earlier stage of development with in particular antibodies directed against IL-12 as described in the PCT application WO 9816248, specific hyaluronans inhibiting the expression of IL-12 and described in the patent application US 2004/097465. There is therefore still a need to develop novel therapies for inflammatory illnesses and other pathologies associated with an over-expression of TNF and/or IL-12.

Phosphatidyl-myo-inositol mannosides (PIMs) are molecules with a low molecular weight (˜2500) known to form part of the mycobacterial wall, which also includes lipoarabinomannanes (LAMs) and lipomannanes (LMs; see FIG. 1). PIMs comprise in general 1 to 4 acylated chains, a glycero-phospho-myo-inositol residue and 1 to 6 mannosylated residues, and can also be synthesised. It is known from the prior art that some LAMs (PILAMs) of rapid-growth and non-virulent species, such as M. smegmatis, are pro-inflammatory molecules simulating the production of TNF and IL-12 (CHATTERJEE, Infect Immun, 1992; GILLERON, J. Biol. Chem., 1997). It has thus been demonstrated that PILAMs activate macrophages by a TLR2-dependent pathway activating the NF-kappaB signalling pathway (MEANS et al., J. Immunol., vol. 163, p: 3920-3927, 1999). Likewise, a pro-inflammatory action of LMs has also been revealed, in particular LMs of Mycobacterium bovis BCG (QUESNIAUX, J. Immunol., 2004; VIGNAL, J. Immunol., 2003). This pro-inflammatory activity results from an induction of the activation of macrophages and pro-inflammatory cytokines by means of the TLR2 receptor and the MyD88 adapter protein (QUESNIAUX et al., J. Immunol., 2004). After a separation of the mono-, di-, tri- and tetra-acylated forms of LM and M. bovis BCG by an extensive purification, it has been possible to demonstrate that tri- and tetra-acylated LMs exhibit a strong TLR-dependent pro-inflammatory activity (GILLERON et al., Chem. Biol., vol. 13, p: 39-47, 2006). Thus the mycobacterial LM acylation profile represents an additional means of regulating the inflammatory response of the host.

Phosphatidyl-myo-inositol dimannoside (PIM₂) and hexamannoside (PIM₆) are the two most copious classes of PIM in Mycobacterium bovis BCG and Mycobacterium tuberculosis H37Rv. PIM₁, PIM₃, PIM₄ and PIM₅ are observed only in limited quantities, suggesting that they correspond to biosynthetic intermediates. PIMs are synthesised from phosphatidylinositol (PI) by the sequential addition of mannose residues at specific positions. The three genes coding for the mannosyl transferases involved in the addition of the first three units α-Manp are now known. The initiation step is catalysed by the enzyme pimA (KORDULAKOVA et al., J. Biol. Chem. 2002) and consists of the transfer of an α-Manp residue into position 2 of the myo-inositol of the PI in order to form PIM₁, while the addition of a second α-Manp residue on the myo-inositol in position 6 is catalysed by the enzyme pimB (SCHAEFFER et al., J. Biol. Chem., vol. 274, p: 31625-31631, 1999). Elongation next takes place by means of pimC (KREMER et al., Biochem. J., vol. 363, p: 437-447, 2002) in order to obtain PIM₃, by the addition of a third α-Manp residue to the α-Manp unit bonded at 6 to the inositol. It has been possible to determine the structure of the various PIMs (GILLERON et al., 1999; GILLERON et al., 2001; GILLERON et al., 2003). Study of PIMs has also made it possible to characterise the various acylated forms of PIMs (GILLERON et al., quoted above, 2001). Finally, the complete synthesis of PIM₂ and PIM₆ was able to be carried out recently (STADELMAIER et al., Carbohydr. Res., vol. 338, p: 2557-69, 2003; LIU et al., J. Am. Chem. Soc., vol. 128, p: 3638-48, 2006).

Surprisingly and unexpectedly, the inventors have shown that some acylated forms of PIM₂ and PIM₆ inhibit the induction of the pro-inflammatory cytokine response. Thus a first object of the invention consists of a pharmaceutical composition comprising at least one compound of formula (I):

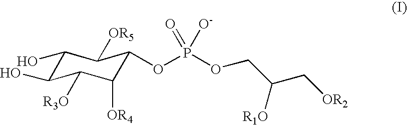

or one of its pharmaceutically acceptable salts in which:
R₁, R₂ and R₃ are independently a hydrogen or an R₇—CO— group where R₇ is an alkyl, alkene or alkyne group, linear, branched or cyclic, comprising 2 to 24 carbon atoms;
R₄ is a hydrogen atom or a mannosyl group substituted in position 6 by an R₆ residue chosen from the group comprising a hydrogen atom and an R₇—CO— group;
R₅ is chosen from the group comprising a hydrogen atom and a mono-, di-, tri-, tetra- or penta-mannosyl.

Advantageously, the mannosyl group or groups are alpha-mannosyl groups. Preferably, the R₅ group is chosen from the group comprising a hydrogen atom, a mono- and a penta-mannosyl. Advantageously again, the R₇ group is a linear alkyl group. Preferably, the R₇ comprises 11 to 21 carbon atoms and particularly preferably 13 to 19 carbon atoms.

The compounds of formula (I) can be obtained simply by a person skilled in the art by purifying PIM from mycobacteria as described in the examples or by chemical synthesis according to the protocol described in STADELMAIER et al. (quoted above, 2003) or in LIU et al. (quoted above, 2006). The pharmaceutically acceptable salts of the compounds of formula (I) are not limited and include, by way of example, inorganic base salts such as alkali metal salts (sodium, lithium, potassium, etc. salts), ammonium salts and organic base salts such as diethylamine, cyclohexamine and amino acid salts.

According to a preferred embodiment, the composition according to the invention comprises at least one compound of formula (I) or one of its pharmaceutically acceptable salts in which formula (I):
One of the R₁, R₂ and R₃ residues is a R₇—CO— group where R₇ is an alkyl, alkene or alkyne group, linear, branched or cyclic, comprising 2 to 24 carbon atoms, and the other two being hydrogen atoms;
R₄ is a hydrogen atom; and
R₅ is a hydrogen atom.

According to a preferred embodiment, the composition according to the invention comprises at least one compound of formula (I) or one of its pharmaceutically acceptable salts in which formula (I):
R₁, R₂ and R₃ are independently a hydrogen or an R₇—CO— group where R₇ is an alkyl, alkene or alkyne group, linear, branched or cyclic, comprising 2 to 24 carbon atoms;
R₄ is a mannosyl group substituted in position 6 by an R₆ residue chosen from the group comprising a hydrogen atom and an R₇—CO— group; and
R₅ is a mannosyl; where
One of the R₁, R₂, R₃ and R₆ residues is an R₇—CO— group and the other three residues being hydrogen atoms.

According to a third preferred embodiment, the composition according to the invention comprises at least one compound of formula (I) or one of its pharmaceutically acceptable salts, in which formula (I):
R₁, R₂ and R₃ are independently a hydrogen or an R₇—CO— group where R₇ is an alkyl, alkene or alkyne group, linear, branched or cyclic, comprising 2 to 24 carbon atoms;
R₄ is a mannosyl group substituted in position 6 by an R₆ residue chosen from the group comprising a hydrogen atom and an R₇—CO— group; and
R₅ is a penta-mannosyl; where
at least two of the R₁ residues, R₂, R₃ and R₆ residues correspond to an R₇—CO— group.
   Advantageously, two, three or four of the R₁ residues, R₂, R₃ and R₆ residues correspond to an R₇—CO— group.

In the composition according to the invention, the compound of formula (I) can be formulated according to well known methods such as solubilised in a solvent, DMSO, water or a buffer or incorporated in emulsions and microemulsions. The composition according to the invention can also comprise components well known in the pharmaceutical field, such as stabilisers, emulsifiers, tonicity agents, preservatives, colourings, excipients, binders and lubricants in particular.

A second object of the invention consists of the use of a composition as described above for the manufacture of a medication intended for the prevention or treatment of an illness associated with the over-expression of TNF and/or IL-12 in a subject. Subject means a mammal, preferably a human.

Illness associated with the over-expression of TNF and/or IL-12 means:
A) immune or auto-immune illnesses such as rheumatoid polyarthritis, graft rejection, sugar diabetes, disseminated erythematous lupus or Basedow's disease;
B) infections and in particular shocks resulting from chronic or acute infection of bacterial, viral and/or parasitic origin;
C) inflammatory illnesses such as chronic inflammatory illnesses (sarcoidosis, inflammatory abdominal ailments, rheumatoid arthritis, haemorrhagic rectocolitis, Crohn's disease) and vascular inflammatory illnesses (defibrination syndrome, arthrosclerosis, Kawasaki disease);
D) neurodegenerative illnesses such as demyelinising illnesses (multiple sclerosis and acute transverse myelitis), extrapyramidal and cerebellar illnesses (lesions of the corticospinal system and basal ganglia disorders);
E) malign pathologies involving tumours secreting TNF or involving TNF such as leukaemia (acute, myelocytic, lymphocytic or chronic myelodysplastic), lymphoma (Hodgkin's or malign (Burkitt's)); and
F) alcohol-induced hepatitis.

The medication is preferably intended for the prevention or treatment of an inflammatory illness in a subject. The medication can be administered by injection (intravenous, intramuscular, subcutaneous, intracutaneous, etc), by nasal, oral or percutaneous administration or by inhalation. According to the administration mode, the said medication can be prepared in the form of solutions, emulsions, pills, powders, ointment, lotions, gels, suppositories or sprays. In the medication, the concentration of compound (I) or its pharmaceutically acceptable salt is not limited and is preferably between 0.1% and 100% (p/p) and particularly preferably between 0.5% and 20%.

### BRIEF DESCRIPTION OF DRAWINGS

FIG. 1 is a schematic representation of M. tuberculosis envelop; and

FIGS. 2-5 are a set of graphs showing test results.

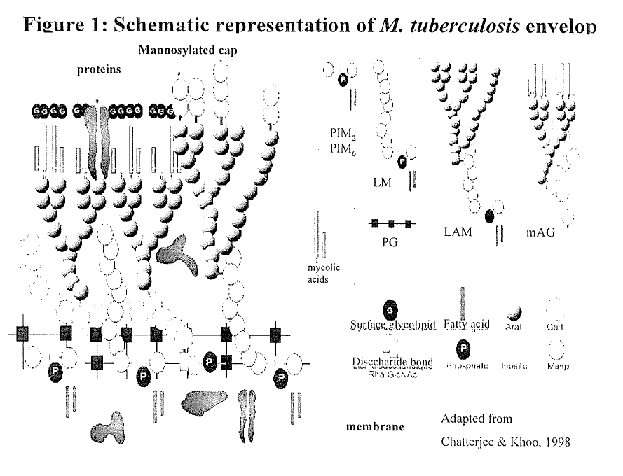

The following examples illustrate the invention and are given non-limitatively.

### Examples

### 1) Purification of the Various Acylated Forms of Phosphatidyl-myo-inositol Di- (PIM₂) And Hexa- (PIM₆) Mannosides

A lipid extract enriched with PIM was obtained by purification of glycolipids of Mycobacterium. Bovis BCG according to the protocol described in VERCELLONE et al. (J. Biol. Chem., vol. 264, p: 7447-7454, 1989) and in GILLERON et al. (J. Biol. Chem., vol. 276, p: 34896-34904, 2001). A lipid extract containing phospholipids insoluble in acetone was then applied to a column of QMA-SPHEROSIL M (BIOSEPRA S.A.) previously balanced by solutions of chloroform, chloroform/methanol (1:1, v/v), methanol in order to elute the neutral compounds. The phospholipids were then eluted in different fractions using organic solvents comprising ammonium acetate:
Fraction A: 750 mg of phospholipids (enriched with phosphatidyl-myo-inositol di-mannosides (PIM₂)) eluted with a chloroform/methanol mixture (1:2, v/v) comprising 0.1 M of ammonium acetate;
Fraction B (subdivided into two fractions): 440 mg of phospholipids (essentially cardiolipids) and 160 mg of phospholipids (mixture of phosphatidyl-myo-inositol di- (PIM₂) and hexa- (PIM₆) mannosides) eluted with a chloroform/methanol mixture (1:2, v/v) comprising 0.2 M of ammonium acetate; and
Fraction C: 55 mg of phospholipids (enriched with phosphatidyl-myo-inositol hexa-mannosides (PIM₆)) eluted with a methanol solution comprising 0.2 M of ammonium acetate.

Successive lyophilisation/re-suspension steps were carried out in order to eliminate the ammonium acetate salts from these various fractions. The various acylated forms were then purified using the fractions obtained.

For the phosphatidyl-myo-inositol hexa-mannosides (PIM₆), 20 mg of phospholipids of fraction C were re-suspended in a solution of 0.1 M ammonium acetate containing 15% (v/v) of propanol-1 by an octyl-sepharose CL-4B column (PHARMACIA) pre-balanced with the same buffer. The column is first of all eluted with 50 ml of balancing buffer and then with a linear gradient of propanol-1 of 15% to 65% (v/v) (each 250 ml) in a solution of 0.1 M ammonium acetate at a rate of 5 ml/h. The fractions were collected every 30 minutes. 20 μ1 of each fraction was dried and subjected to acid hydrolysis (100 μl of 2 M trifluoroacetic acid, 2 hours at 110° C.). The hydrolysates were dried, re-suspended in water and then analysed by high-pH anion exchange chromatography (HPAEC) for their mannose content as described in GILLERON et al. (Mentioned above, 2003). The fractions obtained were grouped together according to their purification profile and repeated lyophilisations eliminated the ammonium acetate salts. A precipitation step with acetone was performed for each fraction in order to eliminate the contaminants issuing from the propanol-1. Finally, 1.2 mg, 1 mg, 7.5 mg and 3 mg of fractions I to IV respectively were obtained.

For the phosphatidyl-myo-inositol dimannosides (PIM₂), 20 mg of phospholipids of fraction A were re-suspended in a solution of 0.1 M ammonium acetate containing 25% (v/v) propanol-1 by CL-4B octyl-sepharose column (PHARMACIA) pre-balanced with the same buffer. The column is first of all eluted with 50 ml of balancing buffer and then with a linear gradient of propanol-1 of 25% to 50% (v/v) (each 125 ml) in a solution of 0.1 M ammonium acetate at a rate of 5 ml/h. The fractions were collected every 15 minutes. 20 μ1 of each fraction was dried and subjected to acid hydrolysis (100 μl of 2 M trifluoroacetic acid, 2 hours at 110° C.). The hydrolysates were dried, re-suspended in water and then analysed by high-pH anion exchange chromatography (HPAEC) for their mannose content as described in GILLERON et al. (quoted above, 2003). The fractions obtained were grouped together according to their purification profile and repeated lyophilisations eliminated the ammonium acetate salts.

### 2) Preparation of Primary Macrophage Cultures

Mice bone marrow cells were obtained from femurs of wild mouse strains C57BL/6 (B6) mice, mice deficient in TLR2 (MICHELSEN et al., J. Biol. Chem., vol. 276, p: 25680-25686, 2001) or in SIGN-R1 (LANOUE et al.), J. Exp. Med., vol. 200, p: 1383-1393, 2004) and control strains corresponding respectively. The cells obtained were cultivated (10⁶/ml) for 7 days in a DMEM environment (DUBECCO) complemented with 20% horse serum and 30% L929 conditioned cell medium (source of M-CSF, MULLER et al., Mol. Med., vol. 2, p: 247-255, 1996). Three days after renewal of the medium, the cell preparation comprises a homogeneous population of macrophages.

### 3) Stimulation of the Macrophages of Wild Mice by LPS in the Presence and Absence of PIM

The macrophages derived from wild mouse bone marrow B6 were cultivated on 96-well culture plates at the rate of 10⁵ cells per well and then stimulated by LPS (100 ng/ml, Escherichia coli, serotype O111 :B4, SIGMA) with or without PIM (6.7 μg/ml). The fractions of PIM used corresponded to the various acylated forms of PIM₆ (Ac₁PIM₆ to Ac₄PIM₆) and to two fractions of PIM₂, a fraction comprising the monoacylated forms of PI and PIM₂ (PIC₁₆ and PIM₂C₁₆) and a fraction comprising the tri- and tetra-acylated forms of PIM₂ (Ac₃PIM₂ and Ac₄PIM₂). All the preparations of lyophilised PIM used were solubilised in DMSO and added to the cultures at a non-cytotoxic final concentration of 1%. After stimulation of 24 hours, the culture supernatants were collected and analysed for their TNF-α and IL-12p40 cytokine content by ELISA (DUOSET) and for their nitrite content by the GRIESS reaction.

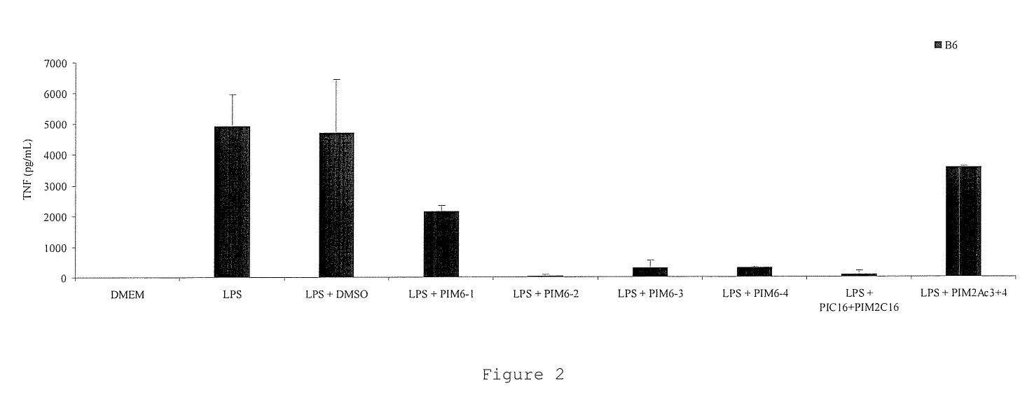

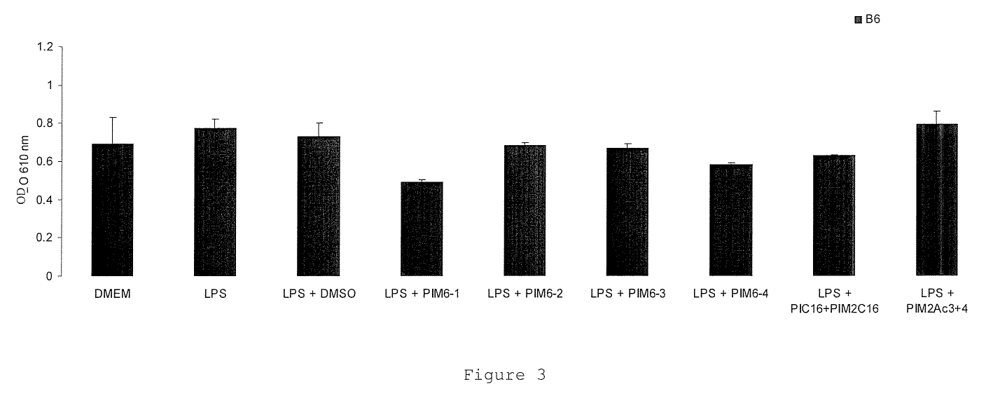

The results show that the di-, tri- and tetra-acylated forms of PIM₆ and the mono-acylated forms of PI and PIM₂ strongly inhibit the synthesis of TNF-α induced in macrophages in the presence of LPS. In addition, the mono-acylated form of PIM₆ and the tri- and tetra-acylated forms of PIM₂ also inhibit this synthesis of TNFα although to a lesser extent, in particular in the case of the tri- and tetra-acylated forms of PIM₂ (FIG. 2). Similar results were obtained for NO and the expression of IL-12p40. An MTT cytotoxicity test performed on the same macrophages in the presence of the various fractions of PIM showed that only the mono-acylated fraction of PIM₆ presents low cytotoxicity for the cells (FIG. 3).

Since preparations of PIM₂ and PIM₆ were identified initially as being stimulators of the secretion of TNF and IL-12p40 by primary cultures of macrophages, non-fractionated preparations of PIM (fraction A to C) were tested on the response induced by LPS at a concentration of 20 μg/ml. The results obtained show no inhibition of the inflammatory response (TNF-α and IL-12p40) of the primary cultures of macrophages induced by LPS in the presence of non-fractionated PIM preparations. This tends to demonstrate that the purity as well as the provenance and nature of the acylated forms of PIM₂ and/or PIM₆ have an influence on the efficacy of the inhibition of the inflammatory response.

### 4) Stimulation of Mouse Macrophages Deficient in TLR2 by LPS in the Presence and Absence of PIM

It had previously been established that the non-fractionated PIM preparations constituted TLR2 agonists (JONES et al., J. Leukoc. Biol., vol. 69, p: 1036-1044, 2001) and that the weak activation of macrophages in the presence of PIM₂ or PIM₆ was dependent on TLR2 (GILLERON et al., cited above, 2003). In order to examine the hypothesis according to which TLR2 would be involved in the anti-inflammatory activity of the acylated fractions of PIM, macrophages derived from mouse bone marrow deficient in TLR2 were cultivated and tested in the presence of LPS with or without a fraction comprising various acylated forms of PIM₂ or PIM₆ as described previously (cf. 3).

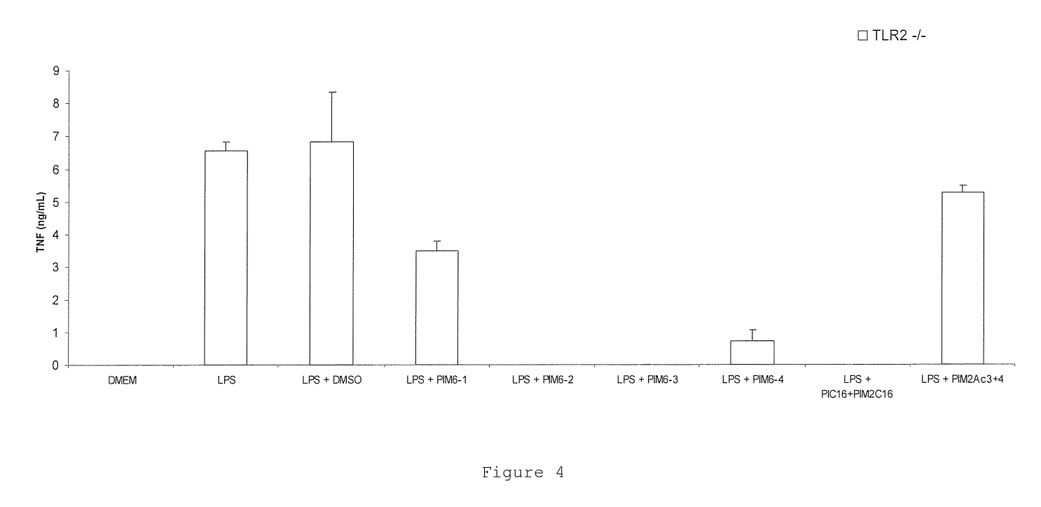

The results show that the di-, tri- and tetra-acylated forms of PIM₆ and the mono-acylated forms of PI and PIM₂ strongly inhibit the synthesis of TNF-α induced in macrophages in the presence of LPS. In addition, the mono-acylated form of PIM₆ and the tri- and tetra-acylated forms of PIM₂ also inhibit this synthesis of TNFα although to a lesser extent, in particular in the case of the tri- and tetra-acylated forms of PIM₂ (FIG. 4). Consequently the anti-inflammatory effect of these fractions is independent of TLR2.

### 5) Stimulation of Macrophages of Mice Deficient in SIGN-R1 by LPS in the Presence and Absence of PIM

The human DC-SIGN receptor is known to be an essential receptor for fixing M. tuberculosis (via the ManLAMs and LMs). In order to examine the hypothesis according to which the mouse receptors of the DC-SIGN family would be involved in the anti-inflammatory activity of acylated fractions of PIM, macrophages derived from the bone marrow of mice deficient in SIGN-R1 were cultivated and tested in the presence of LPS with or without a fraction comprising various acylated forms of PIM₂ or PIM₆as described previously [cf. paragraph 3)].

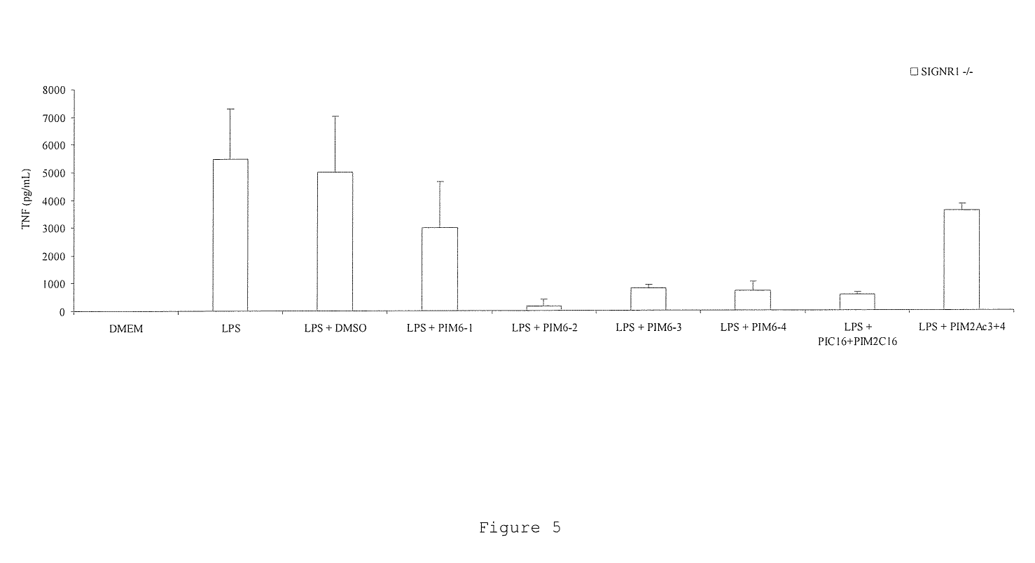

The results show that the di-, tri- and tetra-acylated forms of PIM₆ and the mono-acylated forms of PI and PIM₂ strongly inhibit the synthesis of TNF-α induced in macrophages deficient in SIGN-R1 in the presence of LPS. In addition, the mono-acylated form of PIM₆ and the tri- and tetra-acylated forms of PIM₂ also inhibit this synthesis of TNFα although to a lesser extent, in particular in the case of the tri- and tetra-acylated forms of PIM₂ (FIG. 5). Consequently the anti-inflammatory effect of these fractions is independent of SIGN-R1.

1. A pharmaceutical composition comprising at least one compound of formula (I) comprising: 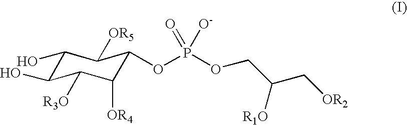
or one of its pharmaceutically acceptable salts in which:
(a) R₁, R₂ and R₃ are independently a hydrogen or an R₇—CO— group where R₇ is an alkyl, alkene or alkyne group, linear, branched or cyclic, comprising 2 to 24 carbon atoms;
(b) R₄ is a hydrogen atom or a mannosyl group substituted in position 6 by an R₆ residue chosen from the group comprising a hydrogen atom and an R₇—CO— group; and
(c) R₅ is chosen from the group comprising a hydrogen atom and a mono-, di-, tri-, tetra- or penta-mannosyl.

2. The composition according to claim 1, wherein the R₇ group is a linear alkyl group.

3. The composition according to claim 1, wherein the R₇ group comprises 11 to 21 carbon atoms.

4. The composition according to claim 1, which composition comprises at least one compound of formula (I) or one of its pharmaceutically acceptable salts, in which formula (I) further comprises:
a. one of the R₁, R₂ and R₃ residues being a R₇—CO— group where R₇ is an alkyl, alkene or alkyne group, linear, branched or cyclic, comprising 2 to 24 carbon atoms, and the other two being hydrogen atoms;
b. R₄ is a hydrogen atom; and
c. R₅ is a hydrogen atom.

5. The composition according to claim 1, which composition comprises at least one compound of formula (I) or one of its pharmaceutically acceptable salts, in which formula (I) further comprises:
(a) R₁, R₂ and R₃ being independently a hydrogen or an R₇—CO— group where R₇ is an alkyl, alkene or alkyne group, linear, branched or cyclic, comprising 2 to 24 carbon atoms;
(b) R₄ is being a mannosyl group substituted in position 6 by an R₆ residue chosen from the group comprising a hydrogen atom and an R₇—CO— group; and
(c) R₅ being a mannosyl;
(d) one of the R₁, R₂, R₃ and R₆ residues being an R₇—CO— group and the other three residues being hydrogen atoms.

6. The composition according to claim 1, which composition comprises at least one compound of formula (I) or one of its pharmaceutically acceptable salts, in which formula (I) comprises:
(a) R₁, R₂ and R₃ being independently a hydrogen or an R₇—CO— group where R₇ is an alkyl, alkene or alkyne group, linear, branched or cyclic, comprising 2 to 24 carbon atoms;
(b) R₄ being a mannosyl group substituted in position 6 by an R₆ residue chosen from the group comprising a hydrogen atom and an R₇—CO— group; and
(c) R₅ being a penta-mannosyl;
(d) at least two of the R₁ residues, R₂, R₃ and R₆ residues corresponding to an R₇—CO— group.

7. The composition according to claim 6, wherein two, three or four of the R₁ residues, R₂, R₃ and R₆ residues correspond to an R₇—CO— group.

8. The composition according to claim 1, further comprising at least one compound chosen from the group comprising stabilisers, emulsifiers, tonicity agents, preservatives, colourings, excipients, binders and lubricants.

9. A use of a composition according to claim 1 for the manufacture of a medication intended for the prevention or treatment of an illness associated with the over-expression of TNF and/or IL-12 in a subject.

10. The use according to claim 9, wherein the illness associated with the over-expression of TNF and/or IL-12 is chosen from the group comprising immune or auto-immune illnesses, infections, inflammatory illnesses, neurodegenerative illnesses, malign pathologies involving tumours secreting TNF or involving TNF and alcohol-induced hepatitis.

11. The use according to claim 10, wherein the illness associated with the over-expression of TNF and/or IL-12 is chosen from inflammatory illnesses.
